(19) United States
(12) Patent Application Publication
(10) Pub. No.: US 2026/0278784 A1
(43) Pub. Date: Sep. 17, 2026

(54) MULTIMODAL DATA FUSION FOR CORONARY HEART DISEASE DIAGNOSTICS AND PROGNOSTICS FROM NON-INVASIVE IMAGING

(71) Applicant: Heartflow, Inc., Mountain View, CA (US)

(72) Inventors: Samuel GERBER, Fayetteville, WV (US); Michiel SCHAAP, Oegstgeest (NL); Andreas SCHUH, Cambridge (GB); Peter Kersten PETERSEN, Palo Alto, CA (US); Matthew SINCLAIR, London (GB); Nan XIAO, San Jose, CA (US); Souma SENGUPTA, Cupertino, CA (US); Timothy A. FONTE, San Francisco, CA (US)

(21) Appl. No.: 19/450,122

(22) Filed: Jan. 15, 2026

Related U.S. Application Data
(60) Provisional application No. 63/745,884, filed on Jan. 16, 2025.

(51) Int. Cl. G06T 7/00; G06T 7/33; G16H 30/40; G16H 50/20

(52) U.S. Cl. CPC G06T 7/0012; G06T 7/344; G16H 30/40; G16H 50/20; G06T 2207/20081; G06T 2207/20221; G06T 2207/30101

(57) ABSTRACT

A method includes receiving data associated with two or more imaging modalities, including an inference imaging modality and a training-only imaging modality. The method includes determining annotations in the training-only imaging modality data, registering data associated with the inference and training-only imaging modalities, and training at least one statistical model to predict features in the inference imaging modality based on the training-only imaging modality data and annotations.

### CROSS-REFERENCE TO RELATED APPLICATIONS

This application claims the benefit of U.S. Provisional Application No. 63/745,884, filed Jan. 16, 2025, entitled “SYSTEMS AND METHODS FOR MULTIMODAL DATA FUSION FOR CORONARY HEART DISEASE DIAGNOSTICS AND PROGNOSTICS FROM NON-INVASIVE IMAGING,” the entire contents of which are incorporated herein by reference.

### FIELD OF INVENTION

The present disclosure relates to medical image processing and analysis for cardiovascular disease assessment, and more particularly to systems and methods for multimodal data fusion that leverage information from multiple imaging modalities to detect, characterize, and quantify coronary artery disease features from non-invasive imaging.

Coronary artery disease (CAD) is the leading cause of mortality among adults in the United States. CAD typically progresses over years or decades, with symptoms often remaining undetected until a severe blockage develops. Current medical understanding suggests that CAD advances through the accumulation of plaque, including non-calcified and calcified plaque, resulting in restricted blood flow. Non-invasive imaging may provide information about the current state of a patient's CAD status, such as plaque burden, stenosis, and myocardial perfusion. This enables healthcare professionals to assess cardiac event risk and make informed treatment decisions without the risk of complications associated with invasive tests.

Non-invasive imaging technologies are limited in the information they can extract about plaque and stenosis due to limitations in resolution and overlapping appearances between the different types of coronary artery tissues and/or surrounding healthy tissue. Systems and methods related to developing statistical algorithms to identify features indicative of CAD across imaging modalities such as Coronary Computed Tomography Angiography (CCTA), Non-Contrast Computed Tomography (NCCT), and Intravascular Ultrasound (IVUS) are described in further detail below. These features may include, but are not limited to, plaque presence and quantity, plaque type classification, stenosis severity and location, and arterial stiffness.

It is to be understood that both the foregoing general description and the following detailed description are exemplary and explanatory and are not restrictive of the disclosure.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an embodiment of the present disclosure, a method is provided. The method may include receiving data associated with two or more imaging modalities. At least one of the imaging modalities may comprise an inference imaging modality and at least one of the imaging modalities may comprise a training-only imaging modality. The method may include determining annotations in the data associated with the training-only imaging modality. The method may include registering data associated with the inference imaging modality and data associated with the training-only imaging modality. The method may include training at least one statistical model to predict features in the inference imaging modality based on the data associated with the training-only imaging modality, the data associated with the inference imaging modality, and the annotations in the data associated with the training-only imaging modality.

According to other embodiments of the present disclosure, the method may include one or more of the following features. The data associated with two or more imaging modalities may comprise data associated with coronary artery disease (CAD). Registering data may comprise image registration, manual co-registration, or statistical model design techniques. Registering data may be either explicit or implicit. Training may be achieved through supervised optimization. The features may include plaque type, plaque geometry, lumen and outer wall geometry, and/or vulnerability score. The imaging modalities may include CCTA, photon-counting CCTA, conventional angiography, OCT, IVUS, CCTA (including PCCT), non-contrast CT, multiple CCTA (including PCCT) images from different acquisitions, 4D flow MRI, and/or applanation tonometry. The features may include stenosis characteristics. The features may include CAD metrics. The method may further comprise an information fusion step that combines features extracted from the two or more of the imaging modalities. The method may further comprise utilizing a second neural network to extract the features from the two or more of the imaging modalities.

According to another embodiment of the present disclosure, the method may further comprise, receiving at least one image associated with a third imaging modality, the at least one statistical model not having been trained with the third imaging modality; an determine features associated with the at least one image using the at least one statistical model.

According to another embodiment of the present disclosure, a system is provided. The system may include one or more processors. The system may include memory storing instructions that, when executed by the one or more processors, cause the system to perform operations. The operations may comprise receiving data associated with two or more imaging modalities. At least one of the imaging modalities may comprise an inference imaging modality and at least one of the imaging modalities may comprise a training-only imaging modality. The operations may comprise determining annotations in the data associated with the training-only imaging modality. The operations may comprise registering data associated with the inference imaging modality and data associated with the training-only imaging modality. The operations may comprise training at least one statistical model to predict features in the inference imaging modality based on the data associated with the training-only imaging modality, the data associated with the inference imaging modality, and the annotations in the data associated with the training-only imaging modality.

According to other embodiments of the present disclosure, the system may include one or more of the following features. The data associated with two or more imaging modalities may comprise data associated with coronary artery disease (CAD). Registering data may comprise image registration, manual co-registration, or statistical model design techniques. Training may be achieved through supervised optimization. The features may include plaque type, plaque geometry, lumen and outer wall geometry, and/or vulnerability score. The imaging modalities may include CCTA, photon-counting CCTA, conventional angiography, OCT, IVUS, CCTA (including PCCT), non-contrast CT, multiple CCTA (including PCCT) images from different acquisitions, 4D flow MRI, and/or applanation tonometry. The operations may further comprise an information fusion step that combines features extracted from the two or more of the imaging modalities. The system may further comprise utilizing a second neural network to extract the features from the two or more of the imaging modalities.

According to another embodiment of the present disclosure, a non-transitory computer-readable medium is provided. The non-transitory computer-readable medium may store instructions that, when executed by one or more processors, cause the one or more processors to perform operations. The operations may comprise receiving data associated with two or more imaging modalities. At least one of the imaging modalities may comprise an inference imaging modality and at least one of the imaging modalities may comprise a training-only imaging modality. The operations may comprise determining annotations in the data associated with the training-only imaging modality. The operations may comprise registering data associated with the inference imaging modality and data associated with the training-only imaging modality. The operations may comprise training at least one statistical model to predict features in the inference imaging modality based on the data associated with the training-only imaging modality, the data associated with the inference imaging modality, and the annotations in the data associated with the training-only imaging modality.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 depicts an exemplary computer environment for performing multimodal data fusion techniques, according to aspects of the present disclosure.

FIG. 2A illustrates a flowchart for a method of multimodal data fusion for coronary heart disease diagnostics and prognostics, according to an embodiment.

FIG. 2B illustrates a flowchart for an inference process, according to aspects of the present disclosure.

FIG. 3 illustrates a flowchart for a method for non-calcified plaque prediction from non-contrast CT, according to an embodiment.

FIG. 4A illustrates a flowchart for a method for CCTA plaque modeling using NCCT during training, according to aspects of the present disclosure.

FIG. 4B illustrates a flowchart for an inference process related to the method of FIG. 4A, according to an embodiment.

FIG. 5A illustrates a flowchart for a training method for CCTA plaque modeling and lumen geometry, according to aspects of the present disclosure.

FIG. 5B illustrates a flowchart for an inference phase of the method of FIG. 5A, according to an embodiment.

FIG. 6A illustrates a flowchart for a training method using multiple CCTA acquisitions, according to aspects of the present disclosure.

FIG. 6B illustrates a flowchart for an inference phase of the method of FIG. 6A, according to an embodiment.

FIG. 7 illustrates a flowchart for a method for predicting artery properties from CT, according to aspects of the present disclosure.

FIG. 8 illustrates a flowchart for a method for multimodal data fusion in medical imaging, according to an embodiment.

FIG. 9 illustrates a block diagram of a system for performing multimodal data fusion techniques, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

Coronary artery disease (CAD) represents a condition where plaque accumulates in coronary arteries over extended periods, often progressing without detectable symptoms until a blockage becomes severe. Non-invasive imaging modalities may provide information about a patient's CAD status, including plaque burden, stenosis, and myocardial perfusion. Such information may enable healthcare professionals to assess cardiac event risk and make treatment decisions without complications associated with invasive procedures.

Non-invasive imaging technologies may be limited in the information that can be extracted about plaque and stenosis due to constraints in resolution and overlapping appearances between different types of coronary artery tissues and surrounding healthy tissue. For example, certain plaque types may exhibit image intensities within ranges that overlap with other tissue types or contrast agents, making differentiation challenging through direct visual inspection or conventional image processing techniques.

The present disclosure describes systems and methods for multimodal data fusion that may address these limitations. The disclosed techniques may involve developing statistical algorithms to identify features indicative of CAD across various imaging modalities. These features may include plaque presence and quantity, plaque type classification, stenosis severity and location, and arterial stiffness, among other characteristics relevant to CAD diagnosis, prognosis, and treatment planning.

The disclosed systems and methods may comprise training and inference phases. During inference, a method may access one or more modalities used in training, and some features may not be readily or fully observable by human inspection in the inference modalities. Statistical image processing algorithms may discern patterns not readily visible to human observers. By utilizing additional input modalities during training, statistical models may be trained to correlate patterns in the inference modalities with annotations from other imaging modalities that may be available during the training phase but not during inference.

In various implementations, the modalities used for statistical model optimization need not correspond to those used during inference. The disclosed techniques may integrate information from multiple input modalities, which may encompass invasive imaging modalities, to construct models for desired inference modalities. This approach may enable the extraction of features from inference modalities that would otherwise be difficult or impossible to detect directly from those modalities alone.

The disclosed multimodal data fusion techniques may leverage the complementary information provided by different imaging modalities. Each imaging modality may have distinct advantages and limitations in characterizing anatomical structures and pathological features. By combining information from multiple modalities during training, the resulting statistical models may learn to identify subtle patterns and relationships that correlate with features of interest, even when those features are not directly observable in the inference modality.

Over fifty percent of heart attacks may occur in asymptomatic patients, and current guidelines may recommend diagnostic imaging tests for symptomatic patients. A calcium scoring scan, which is a non-contrast CT used to identify calcification in arteries, may serve as a fundamental imaging-based diagnostic test for CAD. Calcification may manifest in later stages of CAD. However, non-calcified plaque may pose a risk for a CAD event. Detecting non-calcified plaque may require a contrast-enhanced CT scan, which may be more expensive and may carry additional risks compared to non-contrast CT. Plaque detection and quantification from NCCT may be limited to calcified plaque types using conventional techniques.

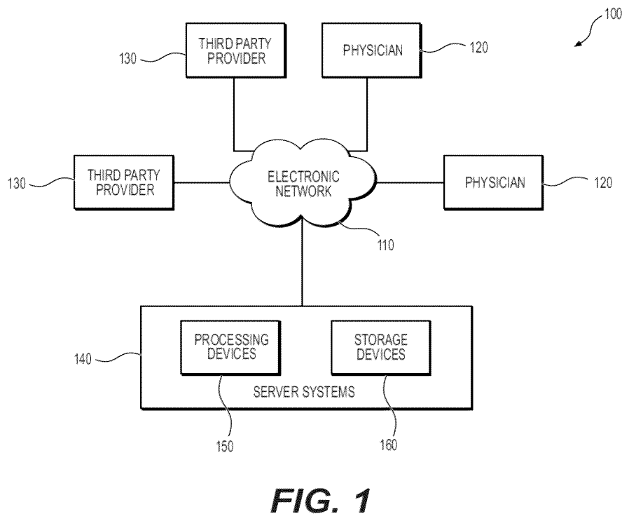

Referring to FIG. 1, an environment 100 for performing the multimodal data fusion techniques described herein is depicted. The environment 100 may include an electronic network 110, such as the Internet, through which various entities may communicate and exchange data. A plurality of physicians 120 and third party providers 130 may be connected to the electronic network 110 through one or more computers, servers, and/or handheld mobile devices.

With continued reference to FIG. 1, each physician 120 and each third party provider 130 may represent a computer system as well as an organization that uses such a system. For example, a physician 120 may be a hospital or a computer system of a hospital. The physicians 120 and/or the third party providers 130 may create or otherwise obtain medical images, such as images of cardiac, vascular, and/or organ systems, of one or more patients. The physicians 120 and/or the third party providers 130 may also obtain any combination of patient-specific information, such as age, medical history, blood pressure, blood viscosity, and other types of patient-specific information.

As further shown in FIG. 1, the physicians 120 and/or the third party providers 130 may transmit patient-specific information to server systems 140 over the electronic network 110. The server systems 140 may include one or more storage devices 160 for storing images and data received from the physicians 120 and/or the third party providers 130. The storage devices 160 may be considered to be components of memory of the server systems 140.

The server systems 140 may also include one or more processing devices 150 for processing images and data stored in the storage devices 160 and for performing any computer-implementable process described in this disclosure. Each of the processing devices 150 may be a processor or a device that includes at least one processor. In some implementations, the server systems 140 may comprise and/or utilize a cloud computing platform with scalable resources for computations and/or data storage, and may run an application for performing methods described in this disclosure on the cloud computing platform. In such implementations, any outputs may be transmitted to another computer system, such as a personal computer, for display and/or storage.

Other examples of computer systems for performing methods of this disclosure may include desktop computers, laptop computers, and mobile computing devices such as tablets and smartphones. A computer system, such as the server systems 140, may include one or more computing devices. If the one or more processors of the computer system are implemented as a plurality of processors, the plurality of processors may be included in a single computing device or distributed among a plurality of computing devices. If a computer system comprises a plurality of computing devices, memory of the computer system may include respective memory of each computing device of the plurality of computing devices.

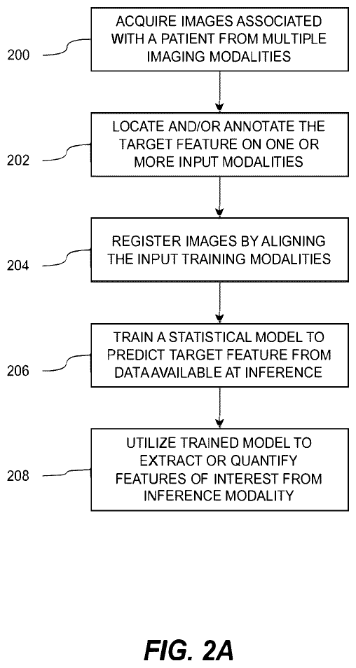

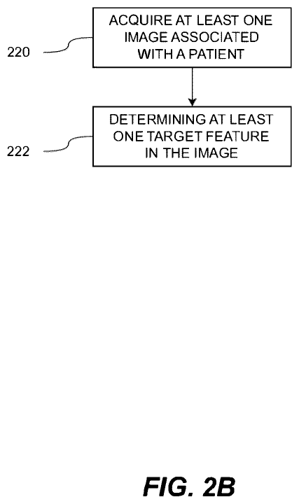

Referring to FIG. 2A, a method for multimodal data fusion for coronary heart disease diagnostics and prognostics is depicted. The method may begin with a step 200, where images associated with a patient are acquired from multiple imaging modalities. The step 200 may comprise receiving data associated with two or more imaging modalities, where at least one of the imaging modalities comprises an inference imaging modality and at least one of the imaging modalities comprises a training-only imaging modality. The data associated with the two or more imaging modalities may comprise data associated with coronary artery disease (CAD). The multiple imaging modalities may be acquired from the same patient, and the acquisition may ensure co-localization of coronary arteries or other structures relevant to the diagnosis or prognosis of CAD across the modalities.

With continued reference to FIG. 2A, the method may proceed to a step 202, where a target feature is located and/or annotated on one or more of the input modalities. The step 202 may comprise determining annotations in the data associated with the training-only imaging modality. The annotations may identify features of interest such as plaque characteristics, stenosis locations, or other CAD-related metrics within the training modality images. The annotation process may be performed manually by trained personnel, automatically through computational techniques, or through a combination of manual and automatic approaches.

Following the step 202, the method may move to a step 204, where images are registered by aligning the input training modalities. The step 204 may comprise registering data associated with the inference imaging modality and data associated with the training-only imaging modality. The registration may facilitate the transfer of annotations between modalities, enabling the correspondence of features identified in the training-only modality to be mapped to corresponding locations in the inference modality. The registration may be performed using image data, using extracted structures such as coronary centerline trees, or a combination of image data and extracted structures.

As further shown in FIG. 2A, the method may advance to a step 206, where a statistical model is trained to predict the target feature from data available at inference. The step 206 may comprise training at least one statistical model to predict features in the inference imaging modality based on the data associated with the training-only imaging modality and the annotations in the data associated with the training-only imaging modality. The training may be achieved through supervised optimization, wherein the statistical model learns to map patterns from the inference modalities to annotations that may originate from imaging modalities available during training but not during inference.

The method may conclude with a step 208, wherein the trained model is utilized to extract or quantify features of interest from the inference modality. During the step 208, the statistical model may apply learned correlations between patterns in the inference modality and annotations from the training-only modality to identify features that may not be readily or fully observable by human inspection in the inference modality. Statistical image processing algorithms may discern patterns not readily visible to human observers by identifying subtle variations in image intensity, texture, spatial relationships, or other characteristics that correlate with the annotated features from the training-only modality.

The method depicted in FIG. 2A may be trained using a fully supervised approach wherein the model learns to delineate, segment, or quantify a feature or structure of interest within the inference modality utilizing annotations from the training modalities. In a fully supervised approach, annotations may be provided for all training samples, and the statistical model may learn to predict these annotations from the corresponding inference modality data. The method may also be trained using a semi-supervised approach with partial annotations. In a semi-supervised approach, annotations may be provided for a subset of training samples, and the statistical model may leverage both annotated and unannotated data to learn feature representations and prediction capabilities.

The multimodal data fusion method depicted in FIG. 2A may enable the extraction of features from inference modalities that would otherwise be difficult to detect directly. By utilizing additional input modalities during training, the statistical models may learn to correlate patterns in the inference modalities with annotations from other imaging modalities. This approach may allow the trained model to identify features in the inference modality based on learned associations, even when those features exhibit overlapping appearances with other tissue types or are otherwise obscured in the inference modality images.

Referring to FIG. 2B, an inference process for the multimodal data fusion method is depicted. The inference process may begin with a step 220, where at least one image associated with a patient is acquired. The step 220 may comprise receiving image data from one or more imaging modalities that correspond to the inference modalities for which the statistical model was trained. The acquired image may be a non-contrast CT image, a contrast-enhanced CT image, or another imaging modality depending on the configuration of the trained statistical model.

With continued reference to FIG. 2B, the inference process may proceed to a step 222, where at least one target feature in the image is determined. During the step 222, the trained statistical model may be applied to the acquired image data to extract or quantify features of interest from the inference modality. The statistical model may utilize learned correlations between patterns in the inference modality and annotations from training-only modalities to identify features such as plaque type, plaque geometry, lumen and outer wall geometry, stenosis characteristics, CAD metrics, or vulnerability scores. The trained model may discern patterns in the inference modality that correlate with features that were annotated in the training-only modality during the training phase, even when those features are not directly observable through human inspection of the inference modality images.

The inference process depicted in FIG. 2B may be configured to operate on different combinations of imaging modalities relative to those used during training. In some implementations, the method may perform inference on the complete set of imaging modalities used during training. For example, a method may be developed where Coronary Computed Tomography Angiography (CCTA) and Non-Contrast Computed Tomography (NCCT) are used during both training and inference. In such implementations, the statistical model may leverage complementary information from multiple modalities during both the training and inference phases.

In other implementations, the method may perform inference on a subset of the imaging modalities used during training. For example, a NCCT-based segmentation model may be trained using co-registered annotations from Intravascular Ultrasound (IVUS), where both NCCT and IVUS are used during training. During inference, the trained model may require NCCT images to obtain a segmentation, without requiring IVUS images.

In further implementations, the method may perform inference on previously unseen modalities using zero-shot learning. A modality-agnostic image segmentation model may be trained on medical images and segmentations from multiple modalities, where one or more inference imaging modalities are not used during training. The modality-agnostic model may learn to extract underlying anatomical features and patterns common across different modalities. The model may learn how different tissues and organs appear in various imaging modalities, recognizing inherent structural relationships and characteristics. This approach may allow the model to infer segmentations in unseen modalities by leveraging knowledge of anatomical structures and variations, even when the model has not been trained on examples from those modalities. For example, a machine learning model could be trained on contrast CT and/or other modalities, but in the inference phase NCCT images could be provided. Although this modality was not trained, segmentations and other features could still be determined.

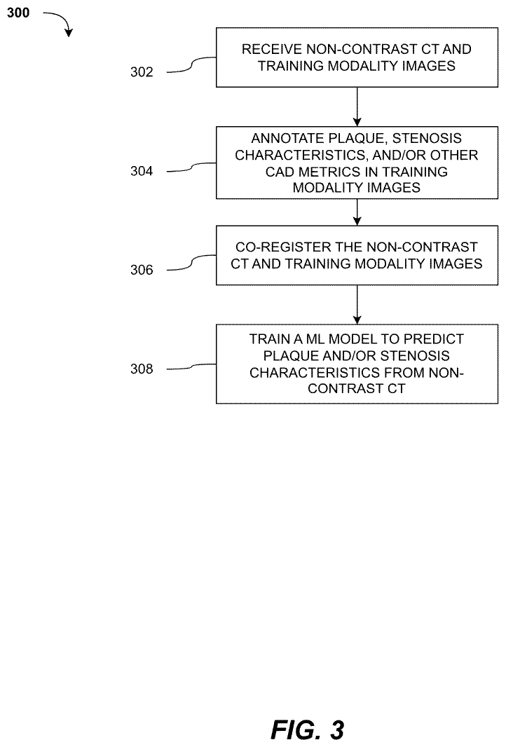

Referring to FIG. 3, a method 300 for non-calcified plaque prediction from non-contrast CT is depicted. The method 300 may begin with a step 302, where non-contrast CT and training modality images are received. The training modalities may include CCTA, photon-counting CCTA, conventional angiography, Optical Coherence Tomography (OCT), or IVUS. The non-contrast CT and training modality images may be acquired from the same patient, and the acquisition may ensure co-localization of coronary arteries or other structures relevant to the diagnosis or prognosis of CAD across the modalities. The joint collection of NCCT and training modalities may enable the correlation of patterns observable in NCCT with features that are more readily identifiable in the training modalities.

The method 300 may proceed to a step 304, where plaque, stenosis characteristics, and/or other CAD metrics in the training modality images are annotated. The annotation may be performed manually by trained personnel, automatically through computational techniques, or through a combination of manual and automatic approaches. The features annotated in the step 304 may include stenosis characteristics such as stenosis severity and location. The features may also include CAD metrics such as plaque burden, plaque distribution, and other quantitative measures relevant to CAD diagnosis and prognosis. The annotations may identify plaque locations and quantities for various plaque types, including calcified plaques and non-calcified plaques. The non-calcified plaque sub-types may include fibrofatty plaques, fibrous plaques, and necrotic plaques. The annotations may also include stenosis locations and severity assessments derived from the training modality images.

As further shown in FIG. 3, the method 300 may move to a step 306, wherein the non-contrast CT and training modality images are co-registered. The co-registration may facilitate the transfer of CAD characteristics between the modalities, enabling annotations identified in the training modalities to be mapped to corresponding locations in the NCCT images. The registration may be performed using image data, using extracted structures such as coronary centerline trees, or a combination of image data and extracted structures. The co-registration may establish spatial correspondence between features in the training modalities and corresponding regions in the NCCT images, enabling the statistical model to learn associations between patterns in NCCT and annotated features from the training modalities.

The method 300 may advance to a step 308, where a machine learning model is trained to predict plaque and/or stenosis characteristics from non-contrast CT. The machine learning model may learn to correlate patterns in the NCCT images with annotations derived from the training modalities. The trained model may predict transferred plaque and stenosis characteristics directly from NCCT without requiring the training modalities during inference. The machine learning model may be trained to predict plaque locations and quantities for fibrous, fibro-fatty, and calcified plaques from NCCT. The machine learning model may also be trained to determine the location and severity of stenoses from NCCT.

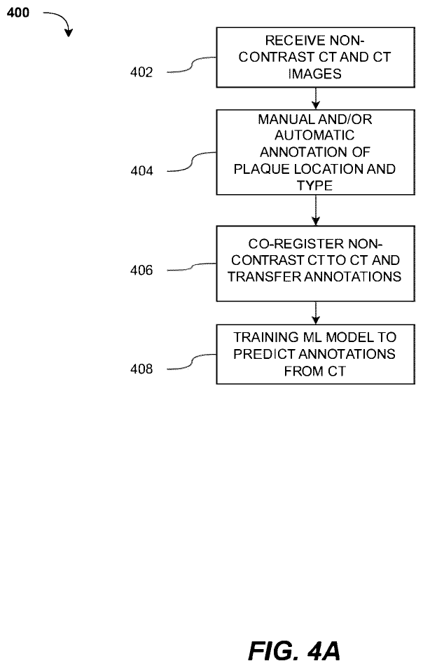

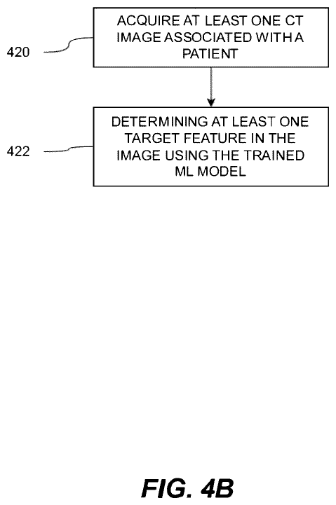

Referring to FIG. 4A, a method 400 for more accurate CCTA plaque modeling and lumen geometry using NCCT during training is depicted. Current automated methods for segmenting coronary plaque and vessel walls may be limited to using a single CCTA acquisition during both training and inference. More accurate segmentations and characterizations from CCTA may be achieved by incorporating information from multiple acquisitions with varying contrast during the training process. The method 400 may extract features of interest for CAD from CCTA by utilizing an NCCT acquisition, such as a calcium scoring scan or a low-dose chest CT, in addition to CCTA images during training.

With continued reference to FIG. 4A, the method 400 may begin with a step 402, where non-contrast CT and CT images are received. The step 402 may comprise receiving CCTA images, which may include Photon Counting CT (PCCT) images, along with an associated NCCT image from the same patient. The joint collection of images from CCTA and NCCT may enable the correlation of patterns observable in CCTA with features that are more readily identifiable when contrast is absent. The NCCT images may provide information about calcified structures that may be obscured or difficult to distinguish in contrast-enhanced images.

The method 400 may proceed to a step 404, which involves manual and/or automatic annotation of plaque location and type in the received images. The annotations may identify features of interest for the diagnosis, prognosis, or treatment planning of CAD. The features may include plaque type, plaque geometry, lumen and outer wall geometry, and/or vulnerability score. The annotations of interest may include location and size of coronary calcified plaque. The annotations of interest may also include location and magnitude of aortic or mitral valve calcification. The annotation process may be performed by trained personnel, through computational techniques, or through a combination of manual and automatic approaches.

As further shown in FIG. 4A, the method 400 may move to a step 406, where co-registration of NCCT to CT is performed and annotations of interest are transferred between the modalities. The co-registration may establish spatial correspondence between features in the NCCT images and corresponding regions in the CCTA images. The transfer of annotations may enable the statistical model to learn associations between patterns in CCTA and annotated features from the NCCT images. The registration may be performed using image data, using extracted structures such as coronary centerline trees, or a combination of image data and extracted structures. In some implementations, registration and segmentation may be performed jointly, wherein the registration model and segmentation model are trained together with joint optimization.

The method 400 may advance to a step 408, where a machine learning model is trained to predict annotations from CT images. The machine learning model may learn to correlate patterns in the CCTA images with annotations derived from the NCCT images. The trained model may be used to extract features of interest from CCTA during inference without requiring NCCT images. The machine learning model may learn to distinguish lumen from calcium in the 300-400 Hounsfield unit (HU) range. In contrast-enhanced CT, the lumen may exhibit image intensities around 300 to 400 HU, while plaque may span ranges from necrotic plaques at approximately −30 HU to calcified plaques above 1000 HU. Plaques within the 300 to 400 HU range may not be readily discernible in contrast-enhanced CT images due to overlap with lumen intensities. By incorporating NCCT during training, the method 400 may provide the training signal for the machine learning model to learn to distinguish lumen from calcium in this overlapping intensity range.

Referring to FIG. 4B, a method 415 for an inference process related to more accurate CCTA plaque modeling and lumen geometry using NCCT during training is depicted. The method 415 represents the application phase where a previously trained model is applied to new patient imaging data to extract or quantify features of interest relevant to coronary artery disease diagnosis, prognosis, or treatment planning. The method 415 may utilize the statistical model trained according to the method 400 described with reference to FIG. 4A, where NCCT was used in conjunction with CCTA during the training phase to develop correlations between patterns in CCTA images and features annotated from NCCT images.

With continued reference to FIG. 4B, the method 415 may begin with a step 420, where at least one CT image associated with a patient is acquired. The step 420 may comprise receiving CCTA image data from the patient. The CCTA image data may include contrast-enhanced CT images of the coronary arteries. The step 420 may not require acquisition of NCCT images, as the trained statistical model may extract features of interest from CCTA images alone during inference. The CCTA images acquired in the step 420 may be transmitted to the server systems 140 over the electronic network 110 for processing by the processing devices 150.

The method 415 may proceed to a step 422, where at least one target feature in the image is determined using the trained machine learning model. During the step 422, the trained statistical model may be applied to the acquired CCTA image data to extract or quantify features of interest from the CCTA images. The features of interest may include plaque type, plaque geometry, lumen and outer wall geometry, and/or vulnerability score. The trained model may utilize learned correlations between patterns in CCTA images and annotations that were derived from NCCT images during the training phase. The step 422 may enable the extraction of features that may not be readily discernible through direct visual inspection of CCTA images alone.

As further shown in FIG. 4B, the method 415 may enable the trained model to distinguish between different tissue types and plaque characteristics in CCTA images without requiring NCCT images during inference. The trained model may have learned during the training phase to identify patterns in CCTA images that correlate with features that are more readily observable in NCCT images. For example, the trained model may distinguish lumen from calcified plaque in the 300-400 HU range, where these structures may exhibit overlapping image intensities in contrast-enhanced CT. By leveraging the correlations learned during training with NCCT, the trained model may identify calcified structures in CCTA images that would otherwise be obscured by the contrast agent in the lumen.

The method 415 may provide features of interest for various clinical applications. The extracted features may be used for stenosis detection by providing accurate lumen wall geometry representations. The extracted features may also be used for fluid simulations for FFR computations, where accurate geometric models of the coronary lumen may improve the accuracy of blood flow and pressure gradient simulations. The method 415 may enable assessment of plaque vulnerability to rupture based on plaque characteristics extracted from the CCTA images. The method 415 may provide these capabilities using CCTA images alone during inference, without requiring the additional NCCT acquisition that was used during the training phase.

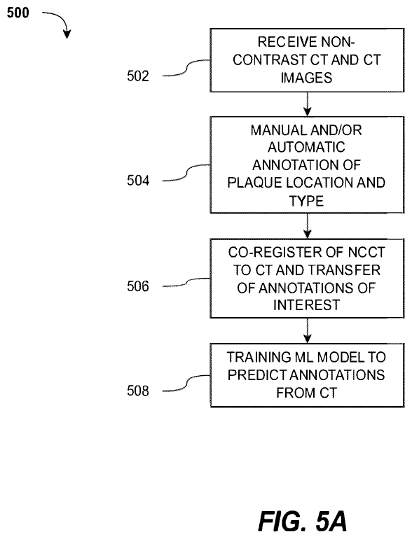

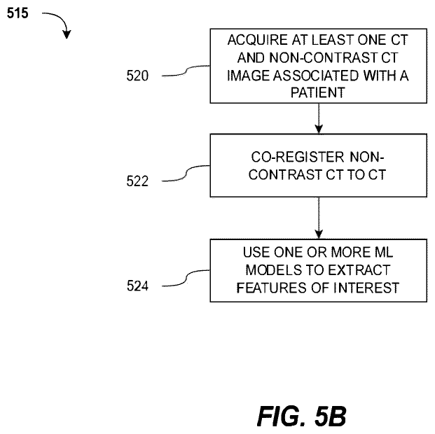

Referring to FIG. 5A, a method 500 for more accurate CCTA plaque modeling and lumen geometry using NCCT during both training and inference is depicted. The method 500 may differ from the method 400 described with reference to FIG. 4A in that the method 500 may utilize both CCTA and NCCT modalities during the inference phase as well as during the training phase. By using both modalities during inference, the method 500 may achieve greater accuracy in feature extraction compared to using CCTA alone.

With continued reference to FIG. 5A, the method 500 may begin with a step 502, where non-contrast CT and CT images are received. The step 502 may comprise receiving CCTA images, which may include PCCT images, along with an associated NCCT image from the same patient. The joint collection of images from CCTA and NCCT may enable the correlation of patterns observable across both modalities with features of interest for CAD diagnosis, prognosis, or treatment planning. The NCCT images may provide complementary information about calcified structures and tissue characteristics that may be obscured or difficult to distinguish in contrast-enhanced images due to overlapping intensity ranges.

The method 500 may proceed to a step 504, which involves manual and/or automatic annotation of plaque location and type in the received images. The annotations may identify features of interest such as plaque type, plaque geometry, lumen and outer wall geometry, and/or vulnerability score. The annotations of interest may include location and size of coronary calcified plaque. The annotations of interest may also include location and size of coronary non-calcified plaque. The annotations may further include location and magnitude of aortic or mitral valve calcification. The features may include plaque vulnerability estimates based on plaque shape, appearance, and location. The annotation process may be performed by trained personnel, through computational techniques, or through a combination of manual and automatic approaches.

As further shown in FIG. 5A, the method 500 may move to a step 506, where co-registration of NCCT to CT is performed along with transfer of annotations of interest. The co-registration may establish spatial correspondence between features in the NCCT images and corresponding regions in the CCTA images. The transfer of annotations may enable the statistical model to learn associations between patterns in both CCTA and NCCT images and the annotated features. The registration may be performed using image data, using extracted structures such as coronary centerline trees, or a combination of image data and extracted structures.

The method 500 may advance to a step 508, where a machine learning model is trained to predict annotations from CT images. The machine learning model may be trained to predict annotations of interest from both CCTA and NCCT images. The training may optimize the statistical model to leverage complementary information from both modalities when predicting features of interest. The machine learning model may learn to correlate patterns observable in CCTA images with patterns observable in NCCT images, and may learn to associate these combined patterns with the annotated features. By training on both modalities, the machine learning model may develop representations that capture information from each modality that may not be available from either modality alone.

Referring to FIG. 5B, a method 515 for an inference phase of more accurate CCTA plaque modeling and lumen geometry using NCCT during both training and inference is depicted. The method 515 represents the application phase where a previously trained model is applied to new patient imaging data to extract or quantify features of interest relevant to coronary artery disease diagnosis, prognosis, or treatment planning. The method 515 may utilize the statistical model trained according to the method 500 described with reference to FIG. 5A, where both CCTA and NCCT were used during the training phase to develop correlations between patterns observable across both modalities and features of interest.

With continued reference to FIG. 5B, the method 515 may begin with a step 520, where at least one CT and non-contrast CT image associated with a patient is acquired. The step 520 may comprise receiving CCTA image data and NCCT image data from the same patient. The CCTA image data may include contrast-enhanced CT images of the coronary arteries. The NCCT image data may include non-contrast CT images such as calcium scoring scans or low-dose chest CT images. The acquisition of both modalities during the step 520 may enable the trained statistical model to leverage complementary information from each modality when extracting features of interest. The CCTA and NCCT images acquired in the step 520 may be transmitted to the server systems 140 over the electronic network 110 for processing by the processing devices 150.

The method 515 may proceed to a step 522, where co-registration of NCCT to CT is performed. The step 522 may establish spatial correspondence between features in the NCCT images and corresponding regions in the CCTA images. The co-registration may align anatomical structures across the two modalities to enable the extraction of features from corresponding locations in each image. The registration may be performed using image data, using extracted structures such as coronary centerline trees, or a combination of image data and extracted structures. The co-registration performed in the step 522 may utilize registration techniques such as image registration, manual co-registration, or statistical model design techniques. The registration may be either explicit, where transformation parameters are computed and applied to align the images, or implicit, where alignment is achieved through learned representations or attention mechanisms.

As further shown in FIG. 5B, the method 515 may advance to a step 524, where one or more machine learning models are used to extract features of interest from the co-registered images. During the step 524, the trained statistical model may be applied to the co-registered CCTA and NCCT image data to extract or quantify features of interest. The features of interest may include plaque type, plaque geometry, lumen and outer wall geometry, vulnerability score, stenosis characteristics, and/or CAD metrics. The trained model may utilize learned correlations between patterns observable in both CCTA and NCCT images and annotations that were derived during the training phase.

The step 524 may be performed using a singular neural network that receives both the co-registered CCTA and NCCT images as input and produces predictions of the features of interest. In such implementations, the singular neural network may have been trained to process both modalities simultaneously and to learn joint representations that capture complementary information from each modality. The singular neural network may output predictions for plaque type, plaque geometry, lumen and outer wall geometry, vulnerability score, or other features of interest based on the combined input from both modalities.

In other implementations, the step 524 may be performed using separate neural networks for the two modalities. A first neural network may be utilized to extract features from the CCTA images, and a second neural network may be utilized to extract features from the NCCT images. The method 515 may further comprise utilizing a second neural network to extract the features from the two or more of the imaging modalities. When separate networks are employed for the different modalities, the step 524 may be succeeded by an information fusion step. The method 515 may further comprise an information fusion step that combines features extracted from the two or more of the imaging modalities. The information fusion step may combine the features extracted from the CCTA images by the first neural network with the features extracted from the NCCT images by the second neural network.

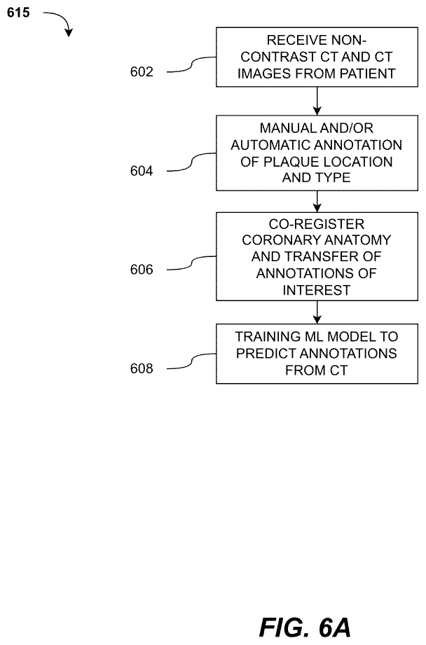

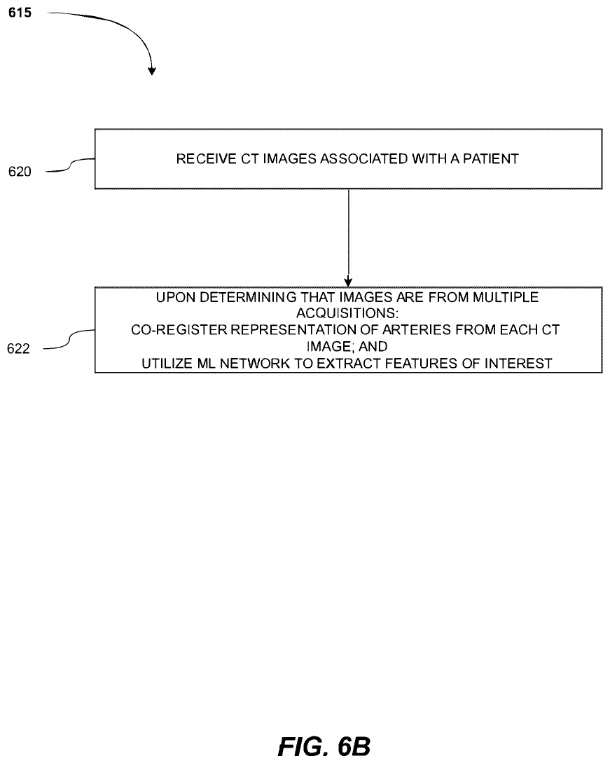

Referring to FIG. 6A, a method 615 for enhanced CCTA plaque modeling and lumen geometry using multiple CCTA acquisitions during a training process is depicted. The method 615 may leverage multiple CCTA images from different acquisitions to develop statistical models capable of extracting features of interest for the diagnosis, prognosis, or treatment planning of coronary artery disease. The multiple CCTA acquisitions may have associated time and intervention between the acquisitions, and this temporal and clinical information may be incorporated into the training process to improve feature extraction accuracy.

With continued reference to FIG. 6A, the method 615 may begin with a step 602, where non-contrast CT and CT images are received from a patient. The step 602 may comprise receiving multiple CCTA images from the same patient, where the multiple CCTA images may be from different acquisitions taken at different times. The multiple CCTA images may include PCCT images. The acquisitions may have associated time intervals between them, ranging from minutes to years. The acquisitions may also have associated intervention information, such as records of medical treatments, lifestyle modifications, or surgical procedures that occurred between the acquisitions. The joint collection of multiple CCTA images from the same patient may enable the correlation of patterns observable across different acquisitions with features of interest for CAD diagnosis, prognosis, or treatment planning.

The method 615 may proceed to a step 604, which involves manual and/or automatic annotation of plaque location and type in the received images. The annotations may identify features of interest such as plaque type, plaque geometry, lumen and outer wall geometry, and/or vulnerability score. The annotations of interest may include location and size of coronary calcified plaque. The annotations of interest may also include location and size of coronary non-calcified plaque. The annotation process may be performed by trained personnel, through computational techniques, or through a combination of manual and automatic approaches. The annotations may be performed on each of the multiple CCTA images, enabling the statistical model to learn from variations in plaque characteristics across different acquisitions.

As further shown in FIG. 6A, the method 615 may move to a step 606, where coronary anatomy is co-registered and annotations of interest are transferred between the imaging modalities. The step 606 may establish spatial correspondence between features in the multiple CCTA images acquired at different times. The co-registration of coronary anatomy may account for differences in patient positioning, cardiac phase, and anatomical changes that may have occurred between acquisitions. The transfer of annotations of interest may enable the statistical model to learn associations between patterns observable in each acquisition and the annotated features.

The step 606 may utilize various techniques for co-registration and annotation transfer. The step of co-registration of coronary anatomy may include modeling the coronary motion to adjust for differences in cardiac phase. The coronary arteries may exhibit motion throughout the cardiac cycle, and images acquired at different cardiac phases may show the coronary anatomy in different positions and configurations. By modeling the coronary motion, the step 606 may align anatomical structures across acquisitions that were captured at different points in the cardiac cycle, enabling accurate correspondence of annotations between the acquisitions.

The step of co-registration of coronary anatomy may also include modeling the disease progression between serial acquisitions. When multiple CCTA acquisitions are separated by extended time intervals, the coronary anatomy may have changed due to disease progression, plaque growth, or plaque regression. By modeling the disease progression between serial acquisitions, the step 606 may account for these anatomical changes when establishing correspondence between features in different acquisitions. The disease progression modeling may utilize information about the time interval between acquisitions and any interventions that occurred during that interval to predict expected changes in plaque characteristics and coronary geometry.

The step of co-registration of coronary anatomy may utilize a canonical, patient-specific coronary vessel tree model. The patient-specific coronary vessel tree model may represent the branching structure and geometry of the coronary arteries for the individual patient. The canonical model may provide a reference framework onto which features from each acquisition may be mapped, enabling correspondence to be established based on anatomical location within the coronary vessel tree. The patient-specific coronary vessel tree model may be constructed from one of the CCTA acquisitions or may be derived from the combination of multiple acquisitions. By mapping features from each acquisition onto the canonical model, the step 606 may establish correspondence between annotations across acquisitions even when the images exhibit differences in cardiac phase, patient positioning, or disease state.

The method 615 may advance to a step 608, where a machine learning model is trained to predict annotations from CT images. The machine learning model may be trained to predict the annotations of interest from the set of CCTA images. The training may optimize the statistical model to leverage information from multiple acquisitions when predicting features of interest. The machine learning model may learn to correlate patterns observable across different acquisitions with the annotated features. The machine learning model may also be trained to predict the annotations of interest from a subset of the images, including from a single image. In such implementations, the machine learning model may learn from the multiple acquisitions during training but may be configured to produce predictions from fewer acquisitions during inference.

The method 615 may use time and intervention between acquisitions as an input to the model for disease progression modeling. The time interval between acquisitions may be provided as an input feature to the machine learning model, enabling the model to learn how plaque characteristics and coronary geometry may change over different time scales. The intervention information, such as records of medical treatments or lifestyle modifications, may also be provided as input features, enabling the model to learn how different interventions may affect disease progression. By incorporating time and intervention information during training, the machine learning model may develop representations that account for temporal dynamics of coronary artery disease and may produce more accurate predictions of current disease state based on historical imaging data.

Referring to FIG. 6B, a method 615 for enhanced CCTA plaque modeling and lumen geometry using multiple CCTA acquisitions during an inference phase is depicted. The method 615 represents the application phase where a previously trained model is applied to new patient imaging data to extract or quantify features of interest relevant to coronary artery disease diagnosis, prognosis, or treatment planning. The method 615 may utilize the statistical model trained according to the training process described with reference to FIG. 6A, where multiple CCTA acquisitions were used during the training phase to develop correlations between patterns observable across different acquisitions and features of interest.

With continued reference to FIG. 6B, the method 615 may begin with a step 620, where CT images associated with a patient are received. The step 620 may comprise receiving CCTA images from one or more acquisitions. The CCTA images may be transmitted to the server systems 140 over the electronic network 110 for processing by the processing devices 150. When images from multiple acquisitions are received, the step 620 may also receive associated time and intervention information describing the interval between acquisitions and any medical treatments or lifestyle modifications that occurred during that interval.

The method 615 may proceed to a step 622, which is performed upon determining that the images are from multiple acquisitions. In the step 622, a geometric representation of the arteries from each CT image is co-registered, and a machine learning network is utilized to extract features of interest from the co-registered images. The method 615 may co-register a geometric representation of the coronary arteries obtained from each CCTA image, such as the lumen centerline, during inference with multiple acquisitions. The co-registration of geometric representations may establish spatial correspondence between anatomical locations across the different acquisitions, enabling the extraction of features from corresponding regions in each image.

As further shown in FIG. 6B, the step 622 may utilize one or more neural networks to extract the features of interest at the corresponding coronary anatomy from the CCTA images. When separate networks are employed, features may be extracted from each image individually, followed by an information fusion step. The information fusion step may combine features extracted from each acquisition to produce final predictions of the features of interest. The time and intervention between the acquisitions may be used in the fusion step to weight or modulate the contributions from each acquisition based on temporal relevance or expected disease progression.

For implementations using a singular neural network, the time and intervention between acquisitions may be provided as an input to the model. The singular neural network may receive the co-registered CCTA images from multiple acquisitions along with the time interval and intervention information, and may produce predictions of the features of interest based on the combined input. By incorporating time and intervention information as input, the singular neural network may account for disease progression when extracting features from multiple acquisitions, enabling more accurate prediction of current disease state based on the temporal sequence of imaging data.

The method 615 may enhance the precision of feature extraction and quantification compared to extracting features from a single CCTA acquisition. By leveraging information from multiple acquisitions, the method 615 may reduce the impact of image noise, artifacts, or suboptimal image quality that may affect individual acquisitions. The method 615 may also leverage temporal information to track disease progression and to produce predictions that account for changes in plaque characteristics over time. The method 615 may enable longitudinal assessment of coronary artery disease by comparing features extracted from serial acquisitions and by modeling expected disease trajectories based on time and intervention information.

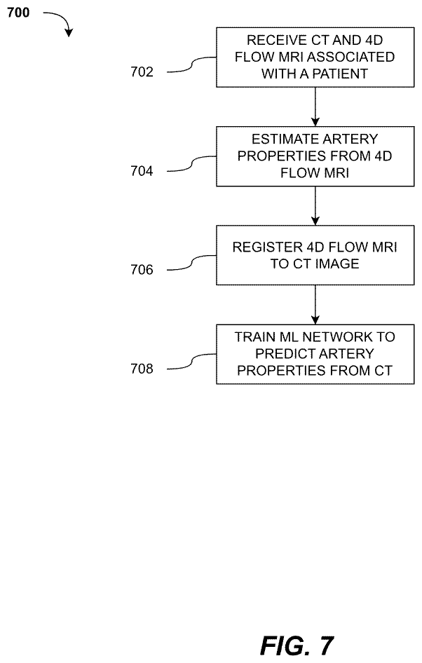

Referring to FIG. 7, a method 700 for predicting artery properties from CT is depicted. The method 700 may estimate artery stiffness, which may serve as an indicator of coronary artery health, directly from CT scans. Artery stiffness may be assessed using 4D flow MRI or applanation tonometry. While 4D flow MRI may demonstrate diagnostic capabilities in identifying severe stable coronary artery disease, the high cost and limited availability of 4D flow MRI may make the modality impractical for widespread screening. By integrating data from both 4D flow MRI and CCTA, the method 700 may develop a model that predicts artery stiffness from CT images. This approach may enable a more accessible and cost-effective means of assessing cardiovascular health in large populations.

With continued reference to FIG. 7, the method 700 may begin with a step 702, where CT and 4D flow MRI associated with a patient are received. The step 702 may comprise receiving CCTA images along with 4D flow MRI data from the same patient. The joint collection of CCTA and 4D flow MRI may enable the correlation of patterns observable in CCTA images with functional measurements derived from 4D flow MRI. The CCTA images may provide anatomical detail of the coronary arteries and surrounding structures, while the 4D flow MRI may provide functional insights including blood flow patterns and vessel wall dynamics. The CCTA and 4D flow MRI data received in the step 702 may be transmitted to the server systems 140 over the electronic network 110 for processing by the processing devices 150.

The method 700 may proceed to a step 704, where artery properties are estimated from the 4D flow MRI. The step 704 may comprise estimating artery stiffness from the 4D flow MRI data. The 4D flow MRI may capture time-resolved, three-dimensional velocity fields of blood flow through the arteries. From these velocity fields, artery stiffness may be derived based on the relationship between blood flow dynamics and vessel wall compliance. The artery stiffness estimates derived in the step 704 may serve as target annotations for training the statistical model to predict artery stiffness from CCTA images. The features extracted in the step 704 may include artery stiffness as a target feature for prediction from CT scans.

As further shown in FIG. 7, the method 700 may move to a step 706, where the 4D flow MRI is registered to the CT image. The step 706 may establish spatial correspondence between anatomical structures in the 4D flow MRI data and corresponding structures in the CCTA images. The registration may align the artery stiffness estimates derived from the 4D flow MRI with corresponding locations in the CCTA images, enabling the statistical model to learn associations between patterns in CCTA images and artery stiffness values. The registration may be performed using image data, using extracted structures such as vessel centerlines, or a combination of image data and extracted structures.

The method 700 may advance to a step 708, where a machine learning network is trained to predict artery properties from CT. The step 708 may comprise training a neural network to predict artery stiffness and 4D flow from CCTA. The machine learning network may learn to correlate patterns in the CCTA images with artery stiffness estimates derived from the 4D flow MRI during the step 704. The trained machine learning network may predict artery stiffness from CCTA images during inference without requiring 4D flow MRI data. The method 700 may predict 4D flow from CCTA in addition to artery stiffness, enabling the estimation of blood flow dynamics from anatomical imaging data alone.

The method 700 may combine the anatomical detail of CT with the functional insights of 4D flow MRI to obtain a more accurate and nuanced picture of artery stiffness. Machine learning algorithms may identify subtle patterns and relationships between image features in CCTA and artery stiffness measurements derived from 4D flow MRI. The method 700 may enable accurate and accessible estimation of artery stiffness from CT images, bypassing the need for additional imaging modalities during inference.

The method 700 may enable risk prediction using CT images by adding estimated artery stiffness as an additional feature for cardiovascular risk assessment. The predicted artery stiffness may be combined with other features extracted from CCTA, such as plaque characteristics and stenosis severity, to produce comprehensive risk predictions. The method 700 may also enable disease monitoring by tracking predicted flows and artery stiffness over time from NCCT or CCTA. The longitudinal monitoring may facilitate tailoring patient-specific treatments, evaluating the effect of different treatments on artery stiffness, and predicting the progression of CAD and the risk of a cardiac event.

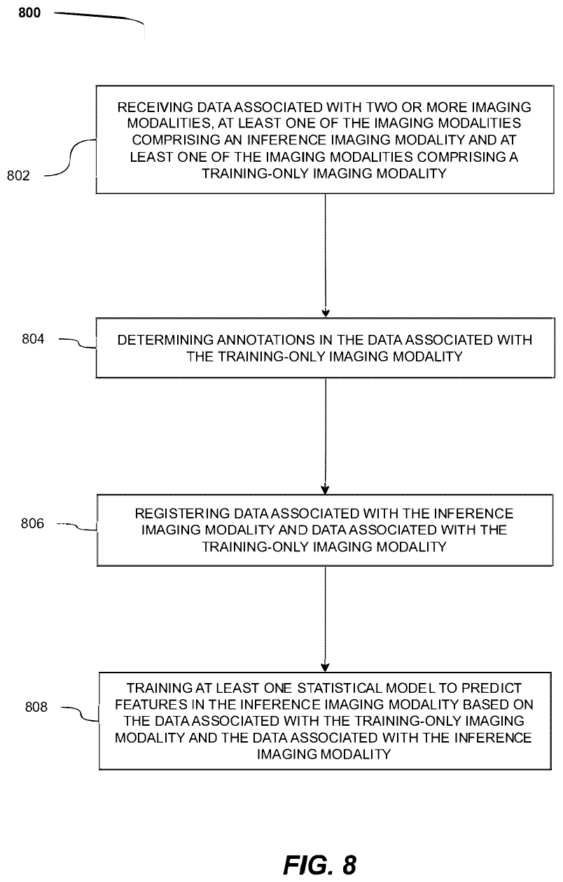

Referring to FIG. 8, a method 800 for multimodal data fusion in medical imaging is depicted. The method 800 may represent a generalized approach for leveraging multiple imaging modalities during training to enable feature prediction from inference modalities that may not directly reveal certain characteristics observable in training-only modalities. The method 800 may be applied to various combinations of imaging modalities and may be configured to extract various features of interest for the diagnosis, prognosis, or treatment planning of coronary artery disease.

With continued reference to FIG. 8, the method 800 may begin with a step 802, where data associated with two or more imaging modalities is received. At least one of the imaging modalities may comprise an inference imaging modality, and at least one of the imaging modalities may comprise a training-only imaging modality. In one embodiment, the inference imaging modality may comprise a non-contrast modality like non-contrast CT, while the training-only imaging modality may comprise contrast-enhanced modality like CCTA. Using these techniques, the system may be trained to produce outputs consistent with contrast-enhanced imaging, even though only non-contrast imaging has been performed. In another technique, the inference imaging modality may comprise a non-invasive modality, while the training-only imaging modality may comprise an invasive imaging modality. Using these techniques, the system may be trained to produce outputs consistent with invasive imaging techniques, even though only non-invasive imaging has been performed. The inference imaging modality may be a modality from which features of interest are to be extracted during the inference phase. The training-only imaging modality may be a modality that provides annotations or complementary information during training but may not be required during inference. The data associated with the two or more imaging modalities may be acquired from the same patient, and the acquisition may ensure co-localization of coronary arteries or other structures relevant to the diagnosis or prognosis of CAD across the modalities. The data received in the step 802 may be transmitted to the server systems 140 over the electronic network 110 for processing by the processing devices 150.

The imaging modalities used in the method 800 may include various non-invasive and invasive imaging modalities. The imaging modalities may include CCTA, photon-counting CCTA, conventional angiography, OCT, IVUS, CCTA including PCCT, non-contrast CT, multiple CCTA including PCCT images from different acquisitions, 4D flow MRI, and/or applanation tonometry. The imaging modalities may also include Cardiac Magnetic Resonance Imaging (CMR) as a non-invasive imaging modality. CMR may provide information about cardiac structure, function, and tissue characteristics without requiring ionizing radiation. The imaging modalities may further include Cinematic phase-contrast magnetic resonance imaging (CineMRI or CINE) as a non-invasive imaging modality. CineMRI may provide time-resolved imaging of cardiac motion and blood flow dynamics. The imaging modalities may also include Positron Emission Tomography (PET) as a non-invasive imaging modality. PET may provide functional and metabolic information about cardiac tissue, including myocardial perfusion and viability assessments.

As further shown in FIG. 8, the method 800 may proceed to a step 804, where annotations in the data associated with the training-only imaging modality are determined. The step 804 may comprise locating and/or annotating target features on one or more of the input modalities. The annotations may identify features of interest such as plaque characteristics, stenosis locations, artery stiffness, or other CAD-related metrics within the training-only modality images. The annotation process may be performed manually by trained personnel, automatically through computational techniques, or through a combination of manual and automatic approaches. The annotations determined in the step 804 may serve as target labels for training the statistical model to predict corresponding features from the inference imaging modality.

The method 800 may move to a step 806, where data associated with the inference imaging modality and data associated with the training-only imaging modality are registered. The step 806 may establish spatial correspondence between features in the training-only imaging modality and corresponding regions in the inference imaging modality. The registration may facilitate the transfer of annotations between modalities, enabling the correspondence of features identified in the training-only modality to be mapped to corresponding locations in the inference modality. The registration may be performed using image data, using extracted structures such as coronary centerline trees, or a combination of image data and extracted structures. The registration may be either explicit, where transformation parameters are computed and applied to align the images, or implicit, where alignment is achieved through learned representations or attention mechanisms.

The method 800 may advance to a step 808, where at least one statistical model is trained to predict features in the inference imaging modality based on the data associated with the training-only imaging modality and the annotations in the data associated with the training-only imaging modality. The step 808 may optimize the statistical model for prediction of target features from the data available at inference. The training may be achieved through supervised optimization, wherein the statistical model learns to map patterns from the inference modalities to annotations that may originate from imaging modalities available during training but not during inference. The statistical model trained in the step 808 may learn to correlate patterns in the inference imaging modality with annotations derived from the training-only imaging modality, enabling the extraction of features that may not be readily or fully observable by human inspection in the inference modality.

The method 800 may enable the selection of different combinations of imaging modalities for training and inference based on clinical requirements and modality availability. For example, the method 800 may be configured with CCTA as the inference imaging modality and IVUS as the training-only imaging modality, enabling the extraction of detailed plaque characteristics from CCTA images by leveraging annotations derived from the higher-resolution IVUS images during training. In another configuration, the method 800 may use non-contrast CT as the inference imaging modality and CCTA as the training-only imaging modality, enabling the prediction of plaque characteristics from non-contrast CT images by leveraging annotations derived from contrast-enhanced imaging during training. The method 800 may also be configured with multiple training-only imaging modalities, such as both NCCT and IVUS, to provide complementary annotations for training the statistical model to extract features from the inference imaging modality.

The methods described herein may employ various registration techniques to align data associated with different imaging modalities. The step of registering data may comprise image registration, manual co-registration, or statistical model design techniques. The step of registering data may be either explicit or implicit depending on the approach employed.

In some implementations, the step of registering data may be performed using separate registration and segmentation models. Under this approach, different imaging modalities may be initially registered using either classical or machine learning-based registration techniques. Subsequently, a segmentation model may be used to predict annotations from the co-registered modality or modalities. The registration may be performed using image data, using extracted structures such as coronary centerline trees, or a combination of image data and extracted structures. Classical registration techniques may include rigid, affine, or deformable registration algorithms that compute transformation parameters to align anatomical structures across modalities. Machine learning-based registration techniques may utilize neural networks trained to predict transformation fields that align input images.

In other implementations, the step of registering data may be performed by jointly training registration and segmentation models. Under this approach, the registration model may consist of differentiable operators that are optimized implicitly to generate transformations for aligning the inference imaging modalities with the training modalities. The joint optimization may incorporate predictions of the segmentation model in the optimization of the registration, and predictions of the registration model may be incorporated into the optimization of the segmentation model. This joint training approach may enable the registration and segmentation models to be optimized together, allowing each model to benefit from the learned representations of the other model.

The step of registering data may establish correspondence via a reference space by mapping features of interest from various modalities onto a unified coordinate system or framework. For coronary arteries, the reference space may include anatomical spaces such as patient-specific 3D atlases. The reference space may also include abstract feature spaces such as latent representations learned through deep learning. Data from each modality may be transformed and mapped onto the chosen reference space, and correspondence may be determined by distance within the designated space. This approach may enable alignment of features across modalities without requiring direct spatial registration of the image volumes.

The step of registering data may utilize attention mechanisms for cross-modality association. Under this approach, input modalities may be tokenized into sub-volumes or sub-patches and embedded into a latent space. Cross-attention may then relate these token embeddings to each other, enabling the model to associate information across modalities without relying on explicit transformation generation. The attention mechanisms may learn to identify corresponding features across modalities based on learned representations rather than spatial alignment, enabling implicit registration through the learned associations between token embeddings from different modalities.

The embodiments described herein may improve computer-implemented analysis of medical images by enabling supervised learning on an inference imaging modality using supervisory signals derived from a different, training-only imaging modality. In some aspects, annotations generated in the training-only modality (e.g., plaque subtype labels, lumen and vessel-wall boundaries, or other anatomical/pathophysiological markers) are transformed into correspondence with inference-modality data via explicit registration, implicit alignment, or mapping into a shared reference representation, thereby producing training targets that are not natively available from the inference modality alone.

In some implementations, the multimodal association is implemented using one or more of: (i) cross-modality registration in image space or feature space; (ii) projection of multimodal data into a common coordinate system or canonical vessel representation; and/or (iii) learned correspondence mechanisms (including attention-based associations) that relate inference-modality patterns to training-only modality annotations. These operations can convert multimodal imaging and annotation inputs into consistent paired examples for training a statistical model to output prediction maps, segmentations, quantitative measurements, and/or structured representations (e.g., centerlines or lumen geometries) from inference-modality inputs.

By structuring the training data in this manner, the disclosed techniques may reduce reliance on invasive intravascular imaging during deployment, while still allowing a model to infer features correlated with intravascular findings. For example, a model may be trained using intravascular imaging modalities such as OCT or IVUS as sources of annotation or supervision, and may then be applied to non-invasive modalities (e.g., CCTA or non-contrast CT) to generate outputs indicative of plaque morphology, vessel-wall characteristics, and/or disease burden.

In some aspects, the disclosed training and correspondence pipeline may improve robustness and reproducibility of coronary artery disease assessment across acquisition protocols by leveraging multi-modality supervision during model development. For instance, the model may learn modality-invariant or modality-consistent features through the shared-reference and correspondence steps described above, which may support more stable estimation of quantities such as plaque composition, stenosis severity, and/or vulnerability-related metrics from inference-modality scans obtained under varying imaging conditions.

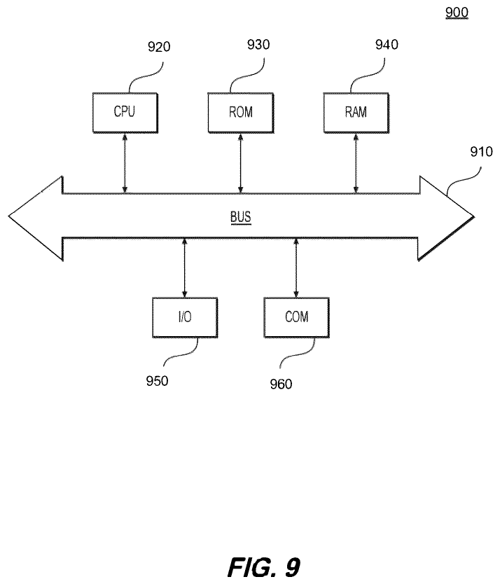

Referring to FIG. 9, a system 900 for performing the multimodal data fusion techniques described herein is depicted. The system 900 may comprise one or more processors and memory storing instructions that, when executed by the one or more processors, cause the system 900 to perform operations for coronary artery disease diagnostics and prognostics. The system 900 may be implemented as part of the server systems 140 described with reference to FIG. 1, or the system 900 may be implemented as a standalone computing device such as a desktop computer, laptop computer, or mobile computing device.

With continued reference to FIG. 9, the system 900 may include a bus 910 that facilitates data transfer and communication between various components of the system 900. The bus 910 may be depicted as a bidirectional connection linking the components of the system 900, indicating that data may flow in both directions between the connected components. The bus 910 may comprise one or more communication pathways that enable the exchange of data, instructions, and control signals between the processor, memory components, and interface components of the system 900.

The system 900 may include a processor 920 connected to the bus 910. The processor 920 may execute instructions and perform computations for implementing the multimodal data fusion methods for coronary artery disease diagnostics and prognostics. The processor 920 may comprise one or more processing units, and the one or more processing units may be implemented as central processing units (CPUs), graphics processing units (GPUs), or specialized processors configured for machine learning computations. The processor 920 may execute instructions stored in memory to perform operations comprising receiving data associated with two or more imaging modalities, determining annotations in the data associated with a training-only imaging modality, registering data associated with an inference imaging modality and data associated with the training-only imaging modality, and training at least one statistical model to predict features in the inference imaging modality.

As further shown in FIG. 9, the system 900 may include a read-only memory 930 connected to the bus 910. The read-only memory 930 may store firmware and permanent data that the processor 920 may access during operation. The read-only memory 930 may contain boot instructions, system configuration data, and other information that may remain unchanged during normal operation of the system 900. The read-only memory 930 may be implemented using non-volatile memory technologies that retain stored data when power is removed from the system 900.

The system 900 may include a random access memory 940 connected to the bus 910. The random access memory 940 may provide temporary storage for data and instructions being actively processed by the processor 920. The random access memory 940 may store imaging data received from the physicians 120 and/or the third party providers 130 during processing operations. The random access memory 940 may also store intermediate results generated during registration, annotation, and statistical model training operations. The random access memory 940 may be implemented using volatile memory technologies that provide rapid read and write access for the processor 920.

With continued reference to FIG. 9, the read-only memory 930 and the random access memory 940 may together comprise memory storing instructions that, when executed by the processor 920, cause the system 900 to perform operations for multimodal data fusion. The memory may store instructions for receiving data associated with two or more imaging modalities, where at least one of the imaging modalities comprises an inference imaging modality and at least one of the imaging modalities comprises a training-only imaging modality. The memory may also store instructions for determining annotations in the data associated with the training-only imaging modality, registering data associated with the inference imaging modality and data associated with the training-only imaging modality, and training at least one statistical model to predict features in the inference imaging modality based on the data associated with the training-only imaging modality and the annotations in the data associated with the training-only imaging modality.

The system 900 may include an input output interface 950 connected to the bus 910. The input output interface 950 may enable the system 900 to receive input data, such as medical images from various imaging modalities, and to output results, such as extracted features of interest or diagnostic predictions. The input output interface 950 may comprise interfaces for connecting to display devices, input devices such as keyboards and pointing devices, and storage devices such as the storage devices 160. The input output interface 950 may enable the processor 920 to receive imaging data from CCTA, photon-counting CCTA, conventional angiography, OCT, IVUS, CCTA including PCCT, non-contrast CT, multiple CCTA including PCCT images from different acquisitions, 4D flow MRI, and/or applanation tonometry. The input output interface 950 may also enable the processor 920 to output extracted features including plaque type, plaque geometry, lumen and outer wall geometry, and/or vulnerability score.

As further shown in FIG. 9, the system 900 may include a communication interface 960 connected to the bus 910. The communication interface 960 may allow the system 900 to communicate with external devices and networks, enabling the transmission and reception of patient-specific information and imaging data. The communication interface 960 may enable the system 900 to connect to the electronic network 110 for receiving imaging data from the physicians 120 and/or the third party providers 130. The communication interface 960 may also enable the system 900 to transmit extracted features, diagnostic predictions, and other results to the physicians 120 and/or the third party providers 130 over the electronic network 110.

The system 900 may execute instructions to perform operations for receiving data associated with two or more imaging modalities. The data associated with the two or more imaging modalities may comprise data associated with coronary artery disease (CAD). The processor 920 may receive imaging data through the communication interface 960 from the electronic network 110 or through the input output interface 950 from connected storage devices. The imaging data may include data from an inference imaging modality and data from a training-only imaging modality acquired from the same patient.

The system 900 may execute instructions to perform operations for determining annotations in the data associated with the training-only imaging modality. The processor 920 may process the training-only imaging modality data to identify and annotate features of interest such as plaque characteristics, stenosis locations, or other CAD-related metrics. The annotation operations may be performed automatically through computational techniques executed by the processor 920, or the annotations may be received through the input output interface 950 from manual annotation performed by trained personnel.

The system 900 may execute instructions to perform operations for registering data associated with the inference imaging modality and data associated with the training-only imaging modality. The registering data may comprise image registration, manual co-registration, or statistical model design techniques. The processor 920 may execute registration algorithms to establish spatial correspondence between features in the training-only imaging modality and corresponding regions in the inference imaging modality. The registration may facilitate the transfer of annotations between modalities, enabling the correspondence of features identified in the training-only modality to be mapped to corresponding locations in the inference modality.

The system 900 may execute instructions to perform operations for training at least one statistical model to predict features in the inference imaging modality based on the data associated with the training-only imaging modality and the annotations in the data associated with the training-only imaging modality. The training may be achieved through supervised optimization, wherein the statistical model learns to map patterns from the inference modalities to annotations that may originate from imaging modalities available during training but not during inference. The processor 920 may execute machine learning training algorithms to optimize parameters of the statistical model based on the registered imaging data and annotations.

The operations performed by the system 900 may further comprise an information fusion step that combines features extracted from the two or more of the imaging modalities. The processor 920 may execute instructions to combine features extracted from different imaging modalities using concatenation, weighted combination, or attention-based fusion techniques. The system 900 may further comprise utilizing a second neural network to extract the features from the two or more of the imaging modalities. The processor 920 may execute a first neural network to extract features from a first imaging modality and may execute a second neural network to extract features from a second imaging modality, with the information fusion step combining the extracted features to produce final predictions.

The instructions executed by the system 900 may be stored on a non-transitory computer-readable medium. The non-transitory computer-readable medium may store instructions that, when executed by one or more processors, cause the one or more processors to perform operations comprising receiving data associated with two or more imaging modalities, where at least one of the imaging modalities comprises an inference imaging modality and at least one of the imaging modalities comprises a training-only imaging modality. The non-transitory computer-readable medium may further store instructions for determining annotations in the data associated with the training-only imaging modality, registering data associated with the inference imaging modality and data associated with the training-only imaging modality, and training at least one statistical model to predict features in the inference imaging modality based on the data associated with the training-only imaging modality and the annotations in the data associated with the training-only imaging modality. The non-transitory computer-readable medium may comprise the read-only memory 930, the random access memory 940, or external storage media connected through the input output interface 950.

A number of implementations have been described. Nevertheless, it will be understood that various modifications may be made without departing from the spirit and scope of the disclosure. Accordingly, other implementations are within the scope of the following claims.

1. A method, comprising:
receiving data associated with two or more imaging modalities, at least one of the imaging modalities comprising an inference imaging modality and at least one of the imaging modalities comprising a training-only imaging modality;
determining annotations in the data associated with the training-only imaging modality;
registering data associated with the inference imaging modality and data associated with the training-only imaging modality; and
training at least one statistical model to predict features in the inference imaging modality based on the data associated with the training-only imaging modality, the data associated with the inference imaging modality, and the annotations in the data associated with the training-only imaging modality.

2. The method of claim 1, wherein the data associated with two or more imaging modalities comprises data associated with coronary artery disease (CAD).

3. The method of claim 1, wherein registering data comprises image registration, manual co-registration, or statistical model design techniques.

4. The method of claim 3, wherein registering data is either explicit or implicit.

5. The method of claim 1, wherein training is achieved through supervised optimization.

6. The method of claim 1, wherein the features include plaque type, plaque geometry, lumen and outer wall geometry, and/or vulnerability score.

7. The method of claim 1, wherein the imaging modalities include CCTA, photon-counting CCTA, conventional angiography, OCT, IVUS, CCTA (including PCCT), non-contrast CT, multiple CCTA (including PCCT) images from different acquisitions, 4D flow MRI, and/or applanation tonometry.

8. The method of claim 1, further comprising:
receiving at least one image associated with a third imaging modality, the at least one statistical model not having been trained with the third imaging modality; and
determining features associated with the at least one image using the at least one statistical model.

9. The method of claim 1, wherein the training-only imaging modality comprises a contrast-enhanced imaging modality, and the inference imaging modality comprises a non-contrast imaging modality.

10. The method of claim 1, further comprising an information fusion step that combines features extracted from the two or more of the imaging modalities.

11. The method of claim 10, further comprising utilizing a second neural network to extract the features from the two or more of the imaging modalities.

12. A system, comprising:
one or more processors; and
memory storing instructions that, when executed by the one or more processors, cause the system to perform operations comprising:
receiving data associated with two or more imaging modalities, at least one of the imaging modalities comprising an inference imaging modality and at least one of the imaging modalities comprising a training-only imaging modality;
determining annotations in the data associated with the training-only imaging modality;
registering data associated with the inference imaging modality and data associated with the training-only imaging modality; and
training at least one statistical model to predict features in the inference imaging modality based on the data associated with the training-only imaging modality, the data associated with the inference imaging modality, and the annotations in the data associated with the training-only imaging modality.

13. The system of claim 12, wherein the data associated with two or more imaging modalities comprises data associated with coronary artery disease (CAD).

14. The system of claim 12, wherein registering data comprises image registration, manual co-registration, or statistical model design techniques.

15. The system of claim 12, wherein training is achieved through supervised optimization.

16. The system of claim 12, wherein the features includes plaque type, plaque geometry, lumen and outer wall geometry, and/or vulnerability score.

17. The system of claim 12, wherein the imaging modalities include CCTA, photon-counting CCTA, conventional angiography, OCT, IVUS, CCTA (including PCCT), non-contrast CT, multiple CCTA (including PCCT) images from different acquisitions, 4D flow MRI, and/or applanation tonometry.

18. The system of claim 12, wherein the operations further comprise an information fusion step that combines features extracted from the two or more of the imaging modalities.

19. The system of claim 18, further comprising utilizing a second neural network to extract the features from the two or more of the imaging modalities.

20. A non-transitory computer-readable medium storing instructions that, when executed by one or more processors, cause the one or more processors to perform operations comprising:
receiving data associated with two or more imaging modalities, at least one of the imaging modalities comprising an inference imaging modality and at least one of the imaging modalities comprising a training-only imaging modality;
determining annotations in the data associated with the training-only imaging modality;
registering data associated with the inference imaging modality and data associated with the training-only imaging modality; and
training at least one statistical model to predict features in the inference imaging modality based on the data associated with the training-only imaging modality, the data associated with the inference imaging modality, and the annotations in the data associated with the training-only imaging modality.
